(19) United States
(12) Patent Application Publication
(10) Pub. No.: US 2026/0157787 A1
(43) Pub. Date: Jun. 11, 2026

(54) Apparatus for applying RF energy to a body

(71) Applicant: Alma Lasers Ltd., Caesarea (IL)

(72) Inventor: Alexander Dverin, Netanya (IL)

(21) Appl. No.: 19/411,634

(22) Filed: Dec. 8, 2025

(30) Foreign Application Priority Data
Dec. 9, 2024 (GB) 2417986.3

(51) Int. Cl. A61B 18/14; A61B 17/00; A61B 18/00

(52) U.S. Cl. CPC A61B 18/14; A61B 2017/00544; A61B 2018/00005; A61B 2018/00077; A61B 2018/0016; A61B 2018/00178; A61B 2018/00184; A61B 2018/00464; A61B 2018/1405; A61B 2018/1467; A61B 2018/1475

(57) ABSTRACT

An apparatus is disclosed for applying radio frequency (RF) energy to a body. The apparatus comprises a housing having a distal surface configured to be held against a surface of the body during operation, an array of applicators mounted in the housing, each applicator being mounted for movement individually relative to the housing between a retracted position, in which the applicator does not project beyond the distal surface, and a deployed position in which the applicator projects beyond the distal surface to contact the body, and a conductor mounted within the housing and connectable to a source of RF energy, wherein each applicator electrically contacts the conductor only when in the deployed position.

### INCORPORATION BY REFERENCE

UK application GB2417986.3, filed Dec. 9, 2024, is hereby incorporated by reference in its entirety.

### THE FIELD OF THE INVENTION

The present invention relates to an apparatus for applying RF energy to a body.

### BACKGROUND OF THE INVENTION

It is well known to apply RF energy to a human body for cosmetic purposes, in particular to dissolve fat. Limitations are placed on the total area that can be treated at any one time by the design of the electrode, or applicator, which contacts the body. Various designs of electrode have been proposed to maximise the area of the body that can be treated with RF energy at any one time, but it is not currently possible to apply RF energy to a large area of a patient, such as the whole of the abdomen, while holding a single electrode in a stationary position. It is therefore current practice to mount the applicator on a handpiece that is moved by the human operator over the area of the body to be treated.

### OBJECT OF THE INVENTION

The present invention seeks to provide a “handsfree” apparatus for applying RF energy to a body, that it to say an apparatus that can enable RF energy to be safely applied to a large area of a patient without requiring human intervention.

### SUMMARY OF THE INVENTION

In accordance with a first aspect of the invention, there is provided an apparatus for applying radio frequency (RF) energy to a body, comprising
a housing having a distal surface configured to be held against a surface of the body during operation,
an array of applicators mounted in the housing, each applicator being mounted for movement individually relative to the housing between a retracted position, in which the applicator does not project beyond the distal surface, and a deployed position in which the applicator projects beyond the distal surface to contact the body, and
a conductor mounted within the housing and connectable to a source of RF energy,
wherein each applicator electrically contacts the conductor only when in the deployed position.

In some embodiments, wherein each applicator is associated with a respective actuator having an output rod that is connected to, and electrically isolated from, the applicator.

Each applicator may have form of an inverted cup mounted on the end of the actuator output rod and having a radially protruding shoulder, and the conductor may be a stationary plate having holes aligned with the actuators. The holes in the conductor plate may have a diameter greater than that of the cups but smaller than that of the shoulders, so that only the shoulder of each applicator makes electrical contact with the plate, and contact between the shoulder and the plate only occurs when the applicator reaches the deployed position.

The end surface of each cup that contacts the body may be spaced from the shoulder by a distance exceeding the distance between the conductor plate and the distal surface of the housing. As a result, during displacement of the cup towards the deployed position, contact between the cup and the body occurs prior to contact between the shoulder and the conductor plate.

In operation, applicators are heated while they are used to apply RF energy to the body of the patient. To avoid the applicators causing discomfort or pain to the patient, they may be cooled by contacting a cooling plate when they are in the retracted position.

In some embodiments each actuator is pneumatically operated and is connected to a source of compressed gas by way of a respective electrically operated supply valve.

In such embodiments, the apparatus may further comprise an electrical control system for operating the supply valves, which control system is configured to activate the valves individually, so that RF energy is applied to the body by only a single applicator at a time.

According to a second aspect of the invention, there is provided an apparatus for applying radio frequency (RF) energy to a body, comprising
a housing having a distal surface configured to be held against a surface of the body during operation,
an array of RF applicators mounted in the housing, each applicator being mounted for movement individually relative to the housing between a retracted position, in which the applicator does not project beyond the distal surface, and a deployed position in which the applicator projects beyond the distal surface to contact the body, and
a control system for causing the applicators to be displaced individually from the retracted position to the deployed position, such that RF energy is supplied to the body though only a single applicator at a time.

In some embodiments, a conductor connectable to a source of RF energy may be mounted within the housing for each applicator to contact electrically only when the actuator is in its deployed position.

### BRIEF DESCRIPTION OF THE DRAWINGS

The invention will now be described further, by way of example, with reference to the accompanying drawings, in which:

FIG. 1 is a perspective view from below of the underside of the housing of an apparatus of the present invention,

FIG. 2 is a perspective view from above of components disposed within the housing of FIG. 1,

FIG. 3 is a perspective view from below of the components shown in FIG. 2, and

FIG. 4 is a section through an applicator module shown in FIGS. 2 and 3.

### Detailed Description of the Drawings

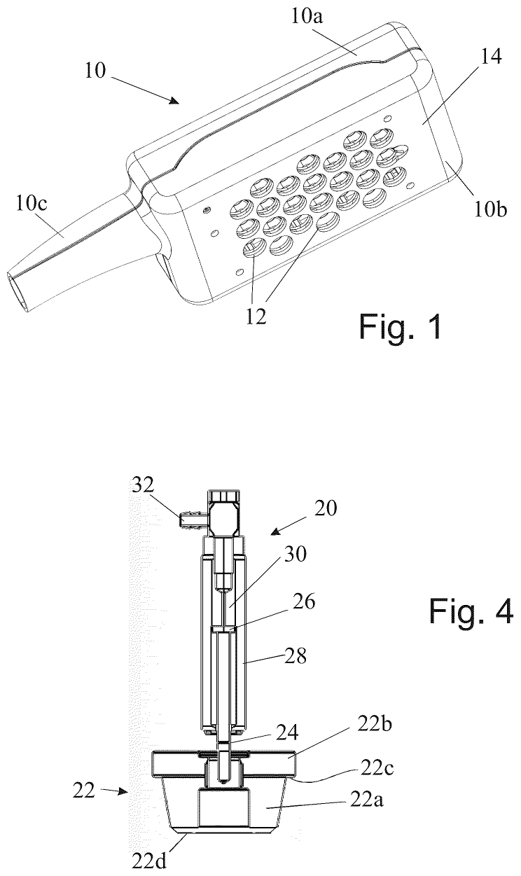

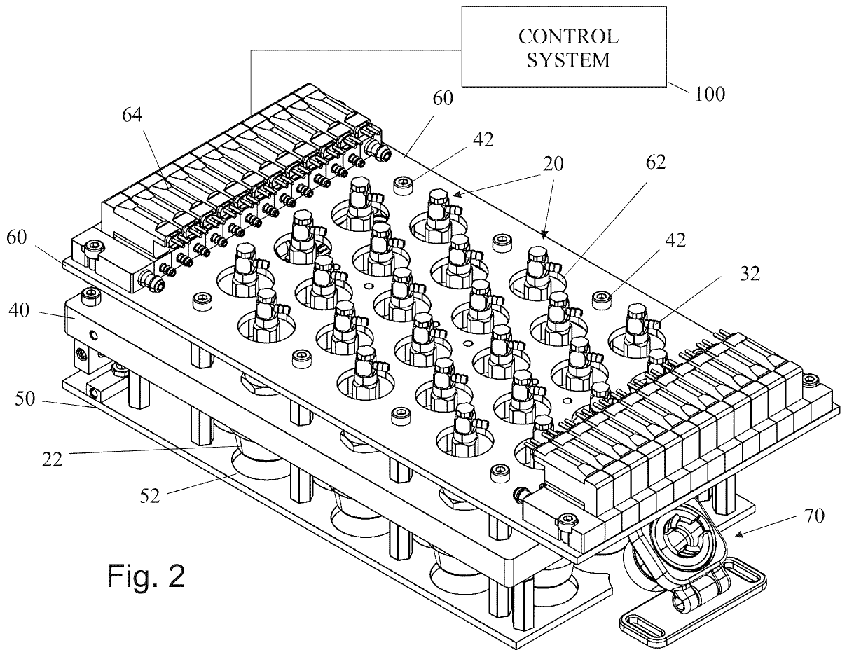

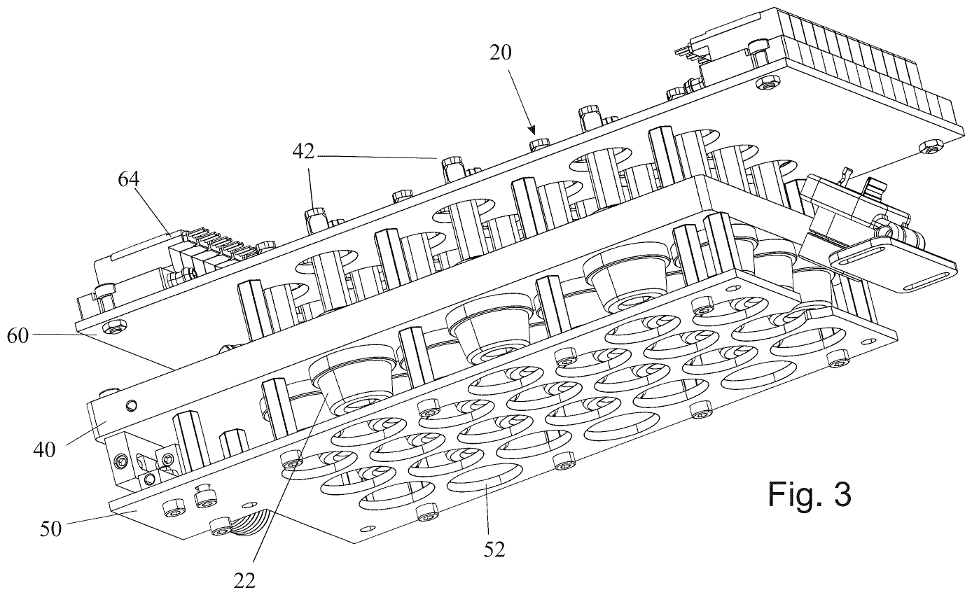

FIG. 1 shows a plastics clamshell housing 10 formed of two halves 10a and 10b that are secured to one another and define an interior chamber housing the various components shown in FIGS. 2 and 3. The housing 10 is intended to be strapped to the body of a patient and the hollow handle part 10c, shown to the left in the drawing, surrounds various wires, pipes and cables to connect the components within the housing to a base unit (not shown).

The housing 10 has a distal surface 14 for contacting the patient's body. The distal surface 14 is provided with an array of holes 12 each aligned with a respective RF applicator disposed within the housing 10. As will be explained below, the applicators are individually movable relative to the housing so that they may all, as shown in FIG. 1, be retracted into the housing. During operation, the applicators are moved on at a time by actuators to a deployed position where they protrude from the housing 10 to contact the body of the patient and apply RF energy. By sequentially actuating the different applicators, it is possible to apply RF energy over an area of the body as large as the housing, without the housing having to be moved manually by an operator.

Referring now to FIGS. 2 to 3, the components arranged in the housing 10 comprise an array of electrode modules 20, one of which is shown in section in FIG. 4. Each electrode module 20 has at its lower end an applicator 22 in the form of an inverted cup 22a mounted on the end of an output rod 24 of a pneumatic actuator. An inverted cup 22a with an annular contact surface 22d is used because an applicator in the form of a flat disk emits most of the RF radiation from its periphery and a cylinder achieves a more uniform energy distribution. If desired, the surface 22d contacting the patient may comprise a plurality of concentric rings instead of only one.

The output rod 24 is connected to a piston 26 disposed with a cylinder 28. The cylinder 28 is held stationary in relation to the housing 10 and when compressed air is introduced into a working chamber 30 disposed above the piston 26, the piston 26 and the rod 24 are forced downwards, as viewed, and cause the applicator 22 to protrude through the respective hole 12 in the housing 10 into contact with the body of the patient. A spring, not shown, arranged in the cylinder 28 and surrounding the piston rod 24, urges the piston 26 upwards towards the retracted position.

The electrode modules are supported on a frame that comprises three plates 40, 50 and 60 that are mechanically secured to one another by screws 42. The screws 42 act through insulators so that the plates are not electrically connected to another.

The plate 50, that is the lowest of the three, is connected through a cable passing through the handle 10c of the housing, to an external RF source located within a control unit of the apparatus. The plate 50 is formed with holes 52 aligned with the applicators 22 of the different electrode modules 20.

The plate 40 serves as a mounting plate for the cylinders 28 of the electrode modules 20. The exterior of the cylinders 28 may for example be screw threaded and each electrode module 20 may be screwed into a respective threaded hole in the mounting plate 40. As well acting as the support for the electrode modules, the plate 40 is cooled and is used to cool the applicators 22 of electrode modules 20 when they are in the retracted position. The plate 40 may be cooled by a circulating fluid or electronically by use of a Peltier effect device.

The uppermost plate 60 serves to support a series of solenoid valves 64, each associated with a respective electrode module 20. The plate 60 also has holes 62 aligned with the electrode modules. Though not shown in the drawings, small pipes or tubes connect a compressed gas input port 32 of each electrode module 20 to a respective one of the solenoid valves 64. When a solenoid valve 64 receives an electrical signal from the control unit of the apparatus, it opens to allow gas from a compressed gas source to flow into the working chamber 30 of the associated electrode module resulting in the applicator 22 being moved to its deployed position. In the absence of a control signal, the solenoid valve vents the working chamber 30 to atmosphere, allowing the spring disposed within each cylinder 28 to urge the piston 26 to the retracted position of the applicator 22.

In the illustrated embodiment of the invention, the applicators 22 are only connected to the RF source when they are in the deployed position and no RF energy reaches either of the plates 40 and 60 at any time. As shown in FIG. 4, the applicator 22 is formed of two parts namely a ceramic disc 22b that is not electrically conductive and is secured to the piston rod 24 and an annular metal part 22a, in the shape of a downwardly tapering hollow cone, that is bonded to the ceramic disc 22b. The ceramic disc also has a metallic coating 22c on its underside that defines a shoulder for contacting the RF plate 50 to create a flow path from the RF plate 50 to the patient through the annular metal part 22a only when the applicator 22 is moved downwards into its deployed position.

At times when the applicator is retracted, the disc 22b rests against the cooled mounting plate 40 to dissipate the heat generated while applying RF energy to the body of the patient. The plate 40 also includes a mounting 70 for a thermometer to allow the temperature of the body to be monitored during treatment.

The axial length of the cup 22a as measured between the contact end surface 22d and the shoulder 22 is greater than the distance between the plate 50 and the distal surface 14 of the housing. As a result, when the contact surface 22d of each electrode is level with the distal surface 14 of the housing to commence making contact with the body of the patient, the shoulder 22c will be still be spaced from the plate 50 and will not therefore be connected to receive RF energy. By the time that contact is made between the applicator 22 and the plate 50 to receive RF energy, the applicator will already be in contact with the body of the patient.

Because of the tapering nature of the cup 22, and the size of the holes 52, it will not make contact with the plate 50 before the shoulder 22c makes contact with the plate 50.

The solenoid valves 64 are connected to a control system 100 which, like the source of compressed air, may be mounted within base unit. The control system 100 applies electrical signals to the solenoid valves 64 to operate the electrode modules 20 individually. In this way, all the RF energy generated by a source mounted in the base unit is applied to the body of the patient through only a single applicator 22 at any one time. The switching between different electrode modules may be done in any desired sequence to achieve even distribution of the RF energy and minimal discomfort to the patient. Furthermore, the control system 100 may allow an operator to determine the duration of each RF application and the time between consecutive applications.

While the invention has been described above by reference to a particular embodiment, it will be appreciated that various modifications may be made without departing from the scope of the invention as set out in the appended claims. For example, in the place of a pneumatic cylinder, a solenoid may be used to move each applicator to and away from its deployed position. Each applicator may adopt a different configuration from an inverted cup, as it may for example comprise a plurality of blunt pins or multiple concentric rings. Furthermore, while the applicator has been described as being cooled only when it its retracted position, it may additionally or alternatively be cooled in the deployed position, such as. by suitable design of the RF conductor plate 50.

1. An apparatus for applying radio frequency (RF) energy to a body, comprising:
a housing having a distal surface configured to be held against a surface of the body during operation,
an array of applicators mounted in the housing, each applicator being mounted for movement individually relative to the housing between a retracted position, in which the applicator does not project beyond the distal surface, and a deployed position in which the applicator projects beyond the distal surface to contact the body, and
a conductor mounted within the housing and connectable to a source of RF energy,
wherein each applicator electrically contacts the conductor only when in the deployed position.

2. Apparatus as claimed in claim 1, wherein each applicator is associated with a respective actuator having an output rod that is connected to, and electrically isolated from, the applicator.

3. Apparatus as claimed in claim 2, wherein each applicator has the form of an inverted cup mounted on the end of the actuator output rod and having a radially protruding shoulder, wherein the conductor is a stationary plate having holes aligned with the actuators, the holes having a diameter greater than that of the cups but smaller than that of the shoulders, whereby only the shoulder of each applicator makes electrical contact with the plate, and contact between the shoulder and the plate only occurs when the applicator reaches the deployed position.

4. Apparatus as claimed in claim 3, wherein each cup has end surface for contacting the body that is spaced from the shoulder by a distance exceeding a distance between the conductor plate and the distal surface of the housing, whereby, during displacement of the cup towards the deployed position, contact between the cup and the body occurs prior to contact between the shoulder and the conductor plate.

5. Apparatus as claimed in claim 3, wherein, in the retracted position, each applicator makes thermal contact with a cooling plate.

6. Apparatus as claimed in claim 3, wherein each actuator is pneumatically operated and is connected to a source of compressed gas by way of a respective electrically operated supply valve.

7. Apparatus as claimed in claim 6, further comprising an electrical control system for operating the supply valves, wherein the control system is configured to activate the valves individually, whereby RF energy is applied to the body by only a single applicator at a time.

8. An apparatus for applying radio frequency (RF) energy to a body, comprising:
a housing having a distal surface configured to be held against a surface of the body during operation,
an array of RF applicators mounted in the housing, each applicator being mounted for movement individually relative to the housing between a retracted position, in which the applicator does not project beyond the distal surface, and a deployed position in which the applicator projects beyond the distal surface to contact the body, and
a control system for causing the applicators to be displaced individually from the retracted position to the deployed position, such that RF energy is supplied to the body through only a single applicator at a time.

9. An apparatus as claimed in claim 8, wherein a conductor connectable to a source of RF energy is mounted within the housing and applicator electrically contacts the conductor only when in the deployed position.
